(19) United States
(12) Patent Application Publication
(10) Pub. No.: US 2022/0013196 A1
(43) Pub. Date: Jan. 13, 2022

(54) METHODS AND COMPOSITIONS FOR ASSESSEMENT OF CONCRETE CARBONATION

(71) Applicant: CARBONCURE TECHNOLOGIES INC., Dartmouth (CA)

(72) Inventors: George Sean MONKMAN, Halifax (CA); Dean Paul FORGERON, Bayside (CA); Brad VICKERS, Bedford (CA)

(21) Appl. No.: 17/369,911

(22) Filed: Jul. 7, 2021

Related U.S. Application Data
(60) Provisional application No. 62/705,617, filed on Jul. 7, 2020.

(51) Int. Cl. G16C 20/20; G01N 33/38; G01N 33/00; G16C 20/10; C04B 40/00

(52) U.S. Cl. CPC G16C 20/20; G01N 33/383; C04B 40/0046; G16C 20/10; C04B 40/0096; G01N 33/004

(57) ABSTRACT

Provided herein are methods and compositions for determining and reporting carbon dioxide sequestered and/or carbon dioxide avoided in operations involving concrete, including concrete raw material transport, concrete production, and concrete use.

### CROSS-REFERENCE TO RELATED APPLICATIONS

This application claims the benefit of U.S. provisional application No. 62/705,617 filed Jul. 7, 2020, which is hereby incorporated by reference as though fully set forth herein.

### BACKGROUND OF THE INVENTION

The use of carbon dioxide in various aspects of concrete batching and use can result in reduction of carbon dioxide emissions, both directly, through direct sequestration of carbon dioxide in the concrete batching, and indirectly, through avoidance of carbon dioxide by, e.g., reducing the amount of cement used in particular batches. Markets have been established to provide carbon credits for such sequestration and avoidance, but a need exists to provide traceable and verifiable information regarding amounts of carbon dioxide offset in given processes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Provided herein are compositions and methods directed to determining amount of carbon, e.g., carbon dioxide, sequestered and/or avoided in the production of concrete. In some cases the compositions and methods can allow for complete or substantially complete traceability from raw materials to batching and then to final use of concrete. In some cases the compositions and methods allow for batch-by-batch assessment of carbon dioxide sequestered and/or avoided, for example at a particular concrete batching facility and/or a plurality of concrete batching facilities and, typically, in real time. Each assessment can be a quantitative value, e.g., kg of carbon dioxide offset, and can be used to obtain carbon credits or equivalents, which can be used in any suitable manner.

In certain embodiments, the compositions and methods utilize a transmitter at a concrete batching facility, where the transmitter transmits relevant information from a treated batch of concrete where some part of the concrete batching operation results in a decrease of carbon dioxide attributable to the batch, compared to an untreated batch. Treatment can include one or both of direct sequestration of carbon dioxide and/or carbon dioxide avoided. Carbon dioxide can be directly sequestered by, e.g., addition of carbon dioxide to the concrete batch while the batch is mixing; carbonation of wash water from batching and transportation operations, for example where some or all of the carbonated wash water is used as mix water for a batch of concrete; carbonation of aggregates used in the batch; and/or carbonation of recycled cement to produce carbonated supplementary cementitious material (SCM). Carbon dioxide may be avoided by, e.g., use of less cement in a treated batch of concrete, from direct reduction in cement quantity and/or replacement of some portion of the cement with supplementary cementitious material that is carbonated and/or with cement in carbonated wash water; avoided carbon dioxide from transportation of cement because less is used in a batch; other avoided transportation offsets as detailed herein. In some cases additional carbon dioxide may be produced in transportation of materials that would otherwise not have been used and this can be entered into calculations as well.

The transmitter is configured to receive and transmit information relevant to treatment of individual batches to offset carbon dioxide. Information may be received from one or more sensors, for example, one or more weight sensors which can include a cement weight sensor, aggregate weight sensor, and/or a water weight sensor, flow sensor if used for water delivery, one or more temperature sensors located in a carbon dioxide delivery system, one or more pressure sensors located in a carbon dioxide delivery system, one or more timers, and/or any other suitable sensors. In some cases a plurality of transmitters, at a plurality of concrete batching sites, may be used, such as at least 2, 3, 4, 5, 7, 10, 15, 20, 25, 30, 40, 50, 70, 100, 150, 200, 300, 400, 500, 700, 1000, 5000, or 10,000 transmitters at at least 2, 3, 4, 5, 7, 10, 15, 20, 25, 30, 40, 50, 70, 100, 150, 200, 300, 400, 500, 700, 1000, 5000, or 10,000 different concrete batching sites, and/or not more than 3, 4, 5, 7, 10, 15, 20, 25, 30, 40, 50, 70, 100, 150, 200, 300, 400, 500, 700, 1000, 5000, 10,000, 50,000, or 100,000 transmitters at at least 3, 4, 5, 7, 10, 15, 20, 25, 30, 40, 50, 70, 100, 150, 200, 300, 400, 500, 700, 1000, 5000, 10,000, 50,000, or 100,000 different concrete batching sites. Transmitters may be wired, wireless, or any other suitable configuration for transmitting information.

In certain embodiments, a first transmitter at a first concrete batching site receives information regarding weight of cement used in a first batch of concrete that is treated with carbon dioxide and transmits that information to a first processor. The weight of the cement used can be compared to historical data for weight of cement in batches with the same mix design but not carbonated. The difference is the amount of cement avoided in the first batch. The processor may also have information regarding the location of a first cement supplier for the first concrete batching site, from which the cement was transported to the batching site; such information can include the amount of carbon dioxide produced in manufacturing a given weight of cement and/or the distance of the first cement supplier from the first batching site. The information may also include average carbon dioxide emissions per unit of distance that the cement is transported, as well as per unit weight, e.g., from trucks, rail, or any other form of transport that is used. The processor can then calculate the amount of carbon dioxide avoided in the first carbonated batch of concrete, based on the weight of cement avoided, the carbon dioxide production per unit weight cement, the weight of cement not transported, and the carbon dioxide production per unit distance and unit weight of transport. The processor may refine such calculations based on any additional aggregate that is used to replace the avoided cement in the first carbonated batch of concrete; such additional aggregate, e.g., fine aggregates such as sand, coarse aggregate, and the like, must be transported from one or more aggregate suppliers to the first concrete batching site and the processor may receive further information from, e.g., one or more weight sensors for aggregates, and determine, based on historical data for uncarbonated batches, the additional amount and kind of aggregate used. The processor may have information regarding location of aggregate producers, amount of carbon dioxide produced during aggregate production and/or transport, and perform calculations similar to those for cement avoided, except that in this case the calculation indicates an additional amount of carbon dioxide produced from additional aggregate used in the first carbonated concrete batch. Any other additional sources of carbon dioxide due to the carbonation process may also be added to the total. The total amount of additional carbon dioxide produced is subtracted from the amount of carbon dioxide avoided due to carbonation of the first concrete mix to give a net amount of carbon dioxide avoided from carbonating the first concrete mix. The process may be carried out in real time or near-real time to provide a figure for net carbon dioxide avoided from carbonating the first concrete mix virtually simultaneously with production of the mix. The figure may be used as is, e.g., provided to a carbon credit market in real time or at some later time, or it may be retained by the processor awaiting, e.g., confirmation of adequate compressive strength for the concrete mix, which can be determined for the mix itself or for the carbonated mix design and process in general. If the latter, there is no need to wait to supply the net carbon dioxide avoided due to carbonation, and the figure may be supplied in real time or close to real time to the market or other source of carbon credits or other value for carbon dioxide offsets.

Additionally or alternatively, the amount of carbon dioxide directly sequestered in the first mix due to carbonation may be determined. Any suitable method may be used to determine total amount of carbon dioxide sequestered in the first batch. For example, in some cases a carbon dioxide container used to provide the carbon dioxide may be weighed, either before and after the first batch, or before and after a series of batches, in which case an average weight reduction per batch may be used. The weight of carbon dioxide added per batch may be modified by efficiency of carbonation for the batches, as described below. The weight sensor can send its information to a second processor, which may be the same or different from the first processor. Additionally or alternatively, a flow rate for carbon dioxide delivery may be determined, e.g., by a flow sensor, and/or by one or more temperature sensors and one or more pressure sensors may be present in the apparatus used to deliver the carbon dioxide to the batching apparatus and be used to determine a flow rate for the carbon dioxide added to the mix. A time sensor may also be used, or time may be estimated from known times of delivery for similar batches. These sensors may send their information to a third processor, which may be the same as or different from the first and second processors; the third processor can calculate a total amount of carbon dioxide added to the first batch of carbonated concrete based on flow rate and time, or number of set time intervals. In some cases the calculation is performed by comparing temperature(s) and pressure(s) with calibration curves. For methods and apparatus for delivering carbon dioxide and calculating flow rates, see, e.g., U.S. Pat. No. 9,376,345 and PCT Patent Publication No. WO2020124054. The total amount of carbon dioxide added may not represent the actual amount of carbon dioxide sequestered in the mix, so generally the processor will also have information regarding the carbonation efficiency of the carbonation process used and can modify the total amount of carbon dioxide delivered by multiplying by efficiency to produce a net amount of carbon dioxide sequestered in the first mix due to carbonation of the mix. This net amount of carbon dioxide sequestered in the first mix due to carbonation of the mix may be added to the net amount of carbon dioxide avoided due to carbonation of the first mix, either at the first, second, or third processors, or at another processor.

Additionally or alternatively, wash water at the first concrete batching site may be carbonated with carbon dioxide. Such carbonated wash water may be sent to waste and/or a portion or all of the carbonated wash water may be used as mix water in subsequent batches of concrete. This may be done without further carbonation in the mix, or the mix may be subject to other carbonation effects, such as delivery of carbon dioxide to the mixing concrete, carbonation of a portion or all of the aggregates used in the mix, carbonation of recycled concrete cement to use as a supplementary cementitious material in the mix, or a combination thereof. For carbonation of wash water, the amount of carbon dioxide sequestered in this process can be determined in a manner similar to that used for addition of carbon dioxide to a concrete mix, or in any other suitable manner. In the case of wash water that is sent to waste, the total amount of carbon dioxide sequestered for a given batch may be calculated by determining gross amount of carbon dioxide added to the wash water and, optionally, multiplying by efficiency of carbonation, to obtain a net amount of carbon dioxide sequestered by wash water. For wash water that is carbonated then added to a concrete mix, the net amount of carbon dioxide sequestered may be determined in a similar manner. Additionally, it is often the case that carbonated wash water provides cement from the wash water to the concrete mix in a form that can replace a portion of the cement that would be used in the mix. In these cases, the amount of carbon dioxide avoided may be calculated as described above, based on the cement avoided by carbonation of wash water; in some cases, the total amount of cement avoided by both carbonating a wet concrete mix and by carbonating wash water that is used in the mix is used as a basis of calculations of carbon avoided, without allocating portions of the avoided carbon to any particular part of the process. Additional information may be obtained regarding carbon dioxide avoided due to the production of carbonated wash water at the facility, which can include any suitable information on carbon dioxide avoidance, such as decreases in energy use to dispose of waste water (due to use in a concrete batch or to other reasons), etc. In certain cases, the system has sensors that provide information to a transmitter regarding flow rates and/or amounts of carbon dioxide added, as described above, though the sensors, e.g., one or more of weight, flow, temperature, and pressure sensors, as well as other sensors used in determining flow rates and amounts for delivery of, e.g., gaseous carbon dioxide, will generally be different than those that determine amount of carbon dioxide added to a mixing batch of concrete. Thus, for a second batch of concrete produced at the first concrete batching facility, the amount of carbon dioxide sequestered from carbonation of wash water used and/or produced in that batch and, in some cases, the amount of carbon dioxide avoided by use of the carbonated wash water, may be calculated in one or more processors which receives signal from the one or more transmitters. The second batch of concrete may be the same as or different from the first batch of concrete. It will be appreciated that for a batches of concrete in which carbonated wash water is used as the mix water, it is important not to double count the carbon dioxide sequestered; that is, carbonated wash water used in one batch is actually produced from wash water from a previous batch, so the amount of carbon dioxide sequestered may be counted for the previous batch, for the present batch, but not for both. For more information on carbonated wash water, see PCT Publication No. WO2018232507.

Additionally or alternatively, aggregates used at the first concrete batching site may be carbonated with carbon dioxide. Any suitable portion of aggregates used in a concrete batch may include recycled concrete aggregates that are carbonated, e.g., hardened concrete that typically is processed to form smaller pieces, then exposed to carbon dioxide, resulting in carbonation of the aggregate. See, e.g., PCT Patent Application No. PCT/IB2020/053953. The amount of carbon dioxide sequestered in this process can be determined in a manner similar to that used for addition of carbon dioxide to a concrete mix, or in any other suitable manner. The total amount of carbon dioxide sequestered for a given batch of recycled aggregates may be calculated by determining gross amount of carbon dioxide added to the recycled aggregates and, optionally, multiplying by efficiency of carbonation, to obtain a net amount of carbon dioxide sequestered by carbonation of the aggregates. For a given batch of concrete, the amount of aggregates that are carbonated recycled aggregates may be determined in any suitable manner, e.g., by weighing. Thus a weight sensor for aggregates may be part of the compositions and methods provided herein, where the weight sensor sends a signal to a transmitter, which in turn sends the signal directly to a processor, or modifies the signal and then sends it to a transmitter. The carbonated aggregates may be used in a third batch of concrete, which can be the same as or different from the first or second batches of concrete.

Additionally or alternatively, waste concrete may be processed to produce a recycled material from the waste concrete that can be used as cement or, more typically, as a supplementary cementitious material (SCM); such processes can involve carbonation of materials at one or more stages in the process; such SCM may be used in one or more concrete batches at the first concrete batching facility. Typically, hardened concrete is processed to separate hardened cement from aggregates; the hardened cement may be further processed to produce particles of appropriate size. Then the particles are exposed to carbon dioxide to carbonate the material, and the resulting carbonated recycled cement may be used in subsequent concrete batches, generally as an SCM. Thus, the process may result both in sequestration of carbon dioxide, which may be calculated as described elsewhere, e.g., by determining total amount of carbon dioxide added and, optionally, multiplying by the efficiency of the process, and in avoided carbon dioxide by substituting part of the cement that would have been used in a concrete batch with the SCM formed by carbonating recycled concrete. In the latter case, the amount of avoided carbon dioxide is calculated in a manner similar to that for avoided carbon dioxide from carbonated a wet mix. Carbon dioxide emitted during transport of the SCM can be factored into the final calculation of net amount of carbon dioxide avoided. The recycled cement from hardened concrete may be used in a fourth batch of concrete, which may be the same as or different from the first, second, or third batches.

Information regarding the source or sources of carbon dioxide used in carbonation processes at the first concrete batching facility may also be provided to one or more processors. Additional information regarding, e.g., the carbon dioxide cost of producing the carbon dioxide and/or transporting it to the site of use may be provided and, in some cases, may be taken into account in determining net amounts of carbon dioxide sequestered and/or avoided. Generally, the source or sources of carbon dioxide come from a source that would have otherwise been emitted to the atmosphere.

In some cases, information from a plurality of concrete batching facilities may be transmitted, via one or more transmitters at each of the facilities, to one or more processors, which may be on-site, remote, or a combination thereof, where the processor or processors determine from the information provided the net amount of carbon offset, e.g., net sequestered carbon dioxide plus net carbon dioxide avoided, for the various batches produced at various facilities that are subject to some form of carbonation. In certain embodiments, at least such as at least 2, 3, 4, 5, 7, 10, 15, 20, 25, 30, 40, 50, 70, 100, 150, 200, 300, 400, 500, 700, 1000, 5000, or 10,000 different concrete batching sites, and/or not more than 3, 4, 5, 7, 10, 15, 20, 25, 30, 40, 50, 70, 100, 150, 200, 300, 400, 500, 700, 1000, 5000, 10,000, 50,000, or 100,000 different concrete batching sites each comprise at least one transmitter, each of which sends information to a processor, such as a local processor, a central processor, or a combination thereof. Information can be sent by a transmitter to a first processor that is local to a concrete batching facility, partially processed, then sent to a central processor that receives information from a plurality of different concrete batching sites. In certain embodiments, each of the concrete batching sites comprises one or more sensors, for example, one or more sensors for weight of cement, one or more sensors for weight of aggregate, one or more temperature sensors associated with a carbon dioxide delivery system, one or more pressure sensors associated with a carbon dioxide delivery system, one or more flow sensors, e.g., water flow sensors or carbon dioxide flow sensors, time sensors, and/or any other suitable sensors, that provide information to the transmitter at each site.

The information is generally transmitted to a processor, which may be on-site or remote, or a combination thereof. More than one processor may be used. A processor can receive information from more than one batching system, such as at least at least 2, 3, 4, 5, 7, 10, 15, 20, 25, 30, 40, 50, 70, 100, 150, 200, 300, 400, 500, 700, 1000, 5000, or 10,000 different concrete batching sites, and/or not more than 3, 4, 5, 7, 10, 15, 20, 25, 30, 40, 50, 70, 100, 150, 200, 300, 400, 500, 700, 1000, 5000, 10,000, 50,000, or 100,000 different concrete batching sites. In some cases the processor is distributed, e.g., the cloud; for the purposes of this description a distributed processor is considered a single processor. Information received by the processor can also include information for raw material producers, e.g. cement and aggregate producers. Such information can include carbon dioxide emission information in the case of cement producers, and location information for both cement and aggregate producers. If additional materials such as carbonated aggregates, carbonated recycled cement, and the like, are used, the locations of these materials may also be entered into the processor. It will be appreciated that much information need only be entered once for a given raw material supplier so long as no relevant parameters are changed batch to batch. The information can also include characteristics of a concrete batch, such as compressive strength at one or more time points, workability (e.g., as assessed by a slump test or similar test), and the like. The processor or processors is configured to assess the information and, for each batch of concrete, to determine a total carbon offset for the batch, that is, the total amount of carbon dioxide directly sequestered and/or avoided in the batch. It can be appreciated that an on-site processor can do some or all of the information processing and/or a remote processor can do some or all of the information processing.

Information regarding one or more of the above characteristics may be provided in visual form. For example, a buyer of a carbon credit that was issued from information at a concrete production site may be provided with a visual representation of one or more of the characteristics. For example, the buyer may be shown a map that shows the sources of various materials used in a concrete batch or batches from which the credit or credits were generated, the concrete batching sites at which concrete was produced, and/or the site or sites where the concrete was or will be used. The amount of carbon dioxide sequestered and/or avoided may be shown at various steps; in some cases, the extra carbon dioxide produced (e.g., from transportation of additional concrete components such as aggregates) is also shown. Thus in some cases the compositions and methods herein are configured to convert data regarding source of carbon dioxide, source of raw materials, concrete batch sites, concrete use sites, and any other suitable information, as well as raw or modified data regarding carbon dioxide sequestration, avoidance, or addition, for one or more batches of concrete from one or more concrete batching sites, into a visual representation of the data such as a map showing geographic locations of sources, batching sites, and/or use sites; and/or visual representation of carbon dioxide sequestered and/or avoided.

The methods and compositions disclosed herein can be used to provide more accurate and up-to-date evaluations of carbon offsets and reporting of these. For example, currently in the concrete industry a concrete manufacturer can supply an Environmental Product Declaration; generally, these are based on industry-wide averages for a finite number of mix design types. That is, they are static averages and do not reflect real-time changes on a batch-to-batch basis or other methods of adjustment based on actual conditions at a given concrete producer, as well as supply and use conditions. In certain embodiments, provided is a dynamic embodied carbon tool, such as an EPD that is updated according to real-world conditions. This allows the development of more accurate EPD and other tools, for example for entire projects development timeline (e.g., design, spec, progress during construction (target to actual) and final project embodied carbon reporting (target to actual)). An adjusted EPD for a concrete manufacturer or other appropriate entity can, for example, start with a current EPD and adjust it based on real-time or close to real-time production data, for example for each mix at a plant or set of plants, to produce a self-adjusting production EPD, for example, for each plant, based on the previous production of that mix in the real world. There can also be real-time or close to real-time feedback along, e.g., a project timeline, to some or all stakeholders, such as owner, AEC, contractors, and the like. The adjustment process can include, in certain embodiments, an initial mix design based on EPD validation, then batch-by-batch (e.g., truck by truck) carbon footprint tracking to adjust the initial EPD, e.g., to develop, an “average production” EPD that accounts for, e.g., production variations. The adjusted “production EPD” can then be used in future submissions. Stakeholder feedback can include providing the concrete producer, contractor, etc., with real-time or close to real-time updates on the project embodied carbon (target to actual), a final report for a project of actual embodied carbon footprint vs. estimated (which can include returned concrete that is not used but billed to the project—so that returned concrete, which can be, e.g., 3-5%, is accounted for in projected accounting), and/or providing producers with options to further reduce carbon footprint and cost savings through, e.g., mix optimization with, for example, theoretical targets. A ranking produced by any suitable method, e.g., AI, can aggregate EPD data across the industry, anonymize the data, and allow a given entity to know their ranking based on the carbon intensity of their different classes of mixes; actions can be suggested to an entity to improve its ranking relative to their peers, e.g., both locally and nationally. This would incentivize producers to further reduce the carbon intensity of their mix designs and/or production schemes. Current EPDs do not incentivize producers for additional carbon savings after the generation of an EPD. EPDs that are continually updating based on real batch data serve as feedback to producers to encourage maintaining or improving their position in the ranking system. Compositions and methods for adjusting, e.g., an EPD to produce a dynamic EPD as opposed to the current, static EPD, can be any appropriate compositions and methods as described herein. Similar considerations can be applied to, e.g., Life Cycle Analyses (LCA) and/or Life Cycle Inventories (LCI).

Thus, provided herein is a system comprising (i) a first concrete production facility, wherein (a) the first concrete production facility comprises a first apparatus to add exogenous carbon dioxide to a component of a first batch of concrete, the first batch of concrete, or both, produced at the facility, (b) a first system to determine information regarding carbon dioxide flow and/or quantity added to the component of the first batch of concrete, carbon dioxide flow and/or quantity added to the first batch of concrete in the first apparatus, a mix design for the first batch of concrete, or a weight of cement used in the first batch of concrete, or a combination thereof, and (c) a first transmitter to transmit the information to a first processor; and (ii) the first processor, wherein the first processor (a) receives inputs from the system for determining information in the first concrete production facility; and (b) processes the inputs to determine an amount of carbon dioxide sequestered and/or offset for the batch of concrete. The component of the first batch of concrete can comprise, e.g., mix water, aggregates, supplementary cementitious material, cement prior to addition to the mix, or a combination thereof, any or all of which may be carbonated by an apparatus configured to expose the component to carbon dioxide. For example, the component can comprise mix water, for example, mix water comprising carbonated wash water from the facility. The system may further include a display apparatus, for example, to provide a representation of the carbon dioxide sequestered and/or avoided, number of carbon dioxide credits, or any other suitable information, to a user. The processor can be configured to further determine a carbon credit or partial credit based, at least in part, on the information from (ii)(b). The system for determining information may include at least one sensor for sensing information regarding carbon dioxide flow and/or quantity added to the component of the first batch of concrete, carbon dioxide flow and/or quantity added to the first batch of concrete in the first apparatus, a mix design for the first batch of concrete, or a weight of cement used in the first batch of concrete. The sensor can comprise, e.g., a weight sensor, a temperature sensor, a pressure sensor, or a combination thereof, in some cases the first system for determining information comprises a weight sensor for sensing the weight of cement added to the first batch of concrete. The first system for determining information may further comprise a human machine interface (HMI) operably connected to the processor, e.g., by a transmitter, which can be the same or different than the transmitter for transmitting other information from the concrete production facility, for entering any additional suitable information necessary or desired for determination of an amount of carbon dioxide sequestered or avoided, e.g., one or more of carbon dioxide flow and/or quantity added to the component of the first batch of concrete, carbon dioxide flow and/or quantity added to the first batch of concrete in the first apparatus, a mix design for the first batch of concrete, or a weight of cement used in the first batch of concrete, or a combination thereof. In some cases, the first processor is remote from the first concrete production facility.

The system may further include one or more systems to provide information to the processor about the source of components of the concrete, e.g., about source of cement or the source or sources of aggregate, for example, distance of source to the concrete batching facility, energy use and/or carbon dioxide production due to transport of the component to the concrete batching facility; information about energy use and/or carbon dioxide produced at the facility due to the production of the first batch of concrete; information about energy use and/or carbon dioxide produced in transporting the first batch of concrete to its job site, and any other information suitable for calculating a total amount of carbon dioxide sequestered and/or avoided in production of the first batch of concrete. This can include historical or other information regarding batches with the same mix design but uncarbonated, for example, weight of cement used in such batches, weight of aggregates used in such batches, and the like. The system may further include a system to provide inputs to the processor regarding market conditions for carbon credits, regulatory information, and the like. The processor my further receive inputs regarding the use of the first batch of concrete, e.g., distance to the job site, type of construction, and the like. Any combination of this additional information may be processed by the processor in determining a net amount of carbon dioxide sequestered and/or avoided, in producing one or more representations of carbon dioxide sequestered and/or avoided or other appropriate representation, or a combination thereof. In certain embodiments the system further determines information regarding carbon dioxide flow and/or quantity added to the component of one or more additional batches of concrete, carbon dioxide flow and/or quantity added to one or more additional batches of concrete in the first apparatus, a mix design for one or more additional batches of concrete, and/or a weight of cement used in one or more additional batches of concrete, produced at the first concrete production facility, or a combination thereof. The processor may be configured to process the information of the first batch and any additional batches in any suitable manner, e.g., aggregating information for some or all batches with a given mix design, aggregating information for some or all batches used in a particular job, such as in a particular construction job, and the like. In certain embodiments the system further comprises additional concrete production facilities each with its own apparatus to add exogenous carbon dioxide to a component of one or more batches of concrete, the one or more batches of concrete, or both, produced at each facility, and each with its own system to determine information regarding carbon dioxide flow and/or quantity added to the component of each of the one or more batches of concrete, carbon dioxide flow and/or quantity added to each of the one or more batches of concrete in the apparatus, a mix design for each of the one or more batches of concrete, or a weight of cement used in each of the one or more batches of concrete, or a combination thereof and each comprising a transmitter to transmit the information to a processor. Systems at the one or more additional plants may include one or more sensors, as detailed for the first plant. The information for each plant may go to the first processor, or another processor, or a combination thereof. The information from the additional facilities can be processed with information from the first facility or independently. In some cases, the facilities are owned, operated, or controlled by a single entity, and appropriate carbon credit calculations may be done based on information from the plurality of facilities, or any suitable combination of a subset of the facilities and/or a subset of batches of concrete produced at the facilities, such as facilities providing concrete to a single job site, and the like.

In certain embodiments, provided is a network comprising (i) a plurality of concrete production facilities, wherein each facility comprises (a) an apparatus to add exogenous carbon dioxide to a component of a first batch of concrete, the first batch of concrete, or both, produced at the facility, (b) a system to determine information regarding carbon dioxide flow and/or quantity added to the component of the first batch of concrete, carbon dioxide flow and/or quantity added to the first batch of concrete in the apparatus, a mix design for the first batch of concrete, or a weight of cement used in the first batch of concrete, and (c) a transmitter to transmit the information to a processor; (ii) the processor, which is configured to (a) receive the information from each of the plurality of concrete production facilities, (b) process the information for each facility to determine an amount of carbon dioxide sequestered and/or avoided for the first batch of concrete produced at each facility. In certain embodiments, the network comprises at least 2, 3, 4, 5, 6, 7, 8, 9, 10, 12, 15, 20, 30, 40, 50, 70, or 100 separate concrete production facilities and/or not more than 3, 4, 5, 6, 7, 8, 9, 10, 12, 15, 20, 30, 40, 50, 70, 100, 200, or 500 separate concrete production facilities. The processor may be any suitable processor. In some cases, one or more of the individual concrete production facilities has one or more intermediate processors that send information to the central processor. In certain embodiments, the processor is configured to determine a carbon credit or partial credit for each first batch of concrete based at least in part on information from (ii). In certain embodiments the concrete production facilities are owned, operated, and/or controlled by a single entity. In certain embodiments the processor is configured to determine a total amount of carbon dioxide sequestered and/or avoided for the sum of at least some of the first batches of concrete produced at each facility. In certain embodiments each concrete production facility comprises (a) at least one sensor to sense a characteristic of materials used or produced in the facility, and/or one or more processes at the facility, and (c) a transmitter to transmit information from the one or more sensors to the remote processor.

In certain embodiments, provided is a method comprising (i) adding exogenous carbon dioxide to a component of a first batch of concrete, the first batch of concrete, or both, produced at a first concrete production facility; (ii) determining information regarding carbon dioxide flow and/or quantity added to the component of the first batch of concrete, carbon dioxide flow and/or quantity added to the first batch of concrete, a mix design for the first batch of concrete, or a weight of cement used in the first batch of concrete; (iii) transmitting the information to a first processor; and (iv) processing the information at the first processor to determine an amount of carbon dioxide sequestered and/or offset for the first batch of concrete. In certain embodiments the component of the first batch of concrete comprises mix water, aggregates, supplementary cementitious material, cement prior to addition to the mix, or a combination thereof. In certain embodiments the component of the first batch of concrete comprises mix water; in some cases the mix water comprises concrete wash water produced at the first concrete batching facility and the apparatus is configured to add carbon dioxide to the wash water to be used as mix water in the batch of concrete. In certain embodiments the first processor (iii) sends the output of step (iv) to a first system to provide a representation of the carbon dioxide sequestered and/or offset to a user. In certain embodiments the processor further determines a carbon credit or partial credit based, at least in part, on the information from step (iv). In certain embodiments the first system for determining information receives information from at least one sensor for sensing information regarding carbon dioxide flow and/or quantity added to the component of the first batch of concrete, carbon dioxide flow and/or quantity added to the first batch of concrete in the first apparatus, a mix design for the first batch of concrete, or a weight of cement used in the first batch of concrete. In certain embodiments the sensor comprises a weight sensor, a temperature sensor, a pressure sensor, or a combination thereof. In certain embodiments the first system for determining information comprises a weight sensor for sensing the weight of cement added to the first batch of concrete. In certain embodiments the first system for determining information comprises a human machine interface (HMI) for entering one or more of carbon dioxide flow and/or quantity added to the component of the first batch of concrete, carbon dioxide flow and/or quantity added to the first batch of concrete in the first apparatus, a mix design for the first batch of concrete, or a weight of cement used in the first batch of concrete. In certain embodiments the first processor is remote from the first concrete production facility. In certain embodiments the method further comprises (i)) adding exogenous carbon dioxide to a component of a first batch of concrete, the first batch of concrete, or both, produced at a second concrete facility, different from the first concrete production facility, (ii) determining information regarding carbon dioxide flow and/or quantity added to the component of the first batch of concrete at the second concrete production facility, carbon dioxide flow and/or quantity added to the first batch of concrete at the second concrete production facility, a mix design for the first batch of concrete at the second concrete production facility, or a weight of cement used in the first batch of concrete at the second concrete production facility, and (c) transmitting the information from the second concrete production facility to a second processor. The first and second processors can be the same or they can be different. In certain embodiments the first and second processors are the same. In certain embodiments the first and second concrete production facilities are owned, operated, or controlled by the same entity. In certain embodiments the first processor further receives inputs regarding market conditions for carbon credits, regulatory information, or a combination thereof. In certain embodiments the first processor further receives inputs regarding the use of the first batch of concrete. In certain embodiments the first processor receives inputs regarding transportation of one or more components of the first batch of concrete, transportation of the first batch of concrete to its site of use, energy use and/or carbon dioxide production at the first concrete production facility during production of the first concrete batch, or a combination thereof. In certain embodiments the information regarding transport comprises information regarding mode of transport, fuel consumption for transport, carbon dioxide emission of fuel consumed, or a combination thereof. Any combination of this additional information may be processed by the processor in determining a net amount of carbon dioxide sequestered and/or avoided, in producing one or more representations of carbon dioxide sequestered and/or avoided or other appropriate representation, or a combination thereof. In certain embodiments the method further comprises (i) adding exogenous carbon dioxide to a component of a second batch of concrete, different from the first batch of concrete, the second batch of concrete, or both, produced at the first concrete production facility; (ii) determining information regarding carbon dioxide flow and/or quantity added to the component of the second batch of concrete, carbon dioxide flow and/or quantity added to the second batch of concrete, a mix design for the second batch of concrete, or a weight of cement used in the second batch of concrete, (iii) transmitting the information to the first processor; and (iv) processing the information at the first processor to determine an amount of carbon dioxide sequestered and/or offset for the second batch of concrete. The method may further include adding exogenous carbon dioxide to any number of additional batches of concrete at the first facility, determining information regarding carbon dioxide flow or quantity, transmitting information to the first processor, and processing the information, as described; for example, at least 1, 2, 3, 4, 5, 6, 7, 8, 9, 10, 12, 15, 20, 30, 40, 50, 70, 100, 200, 500, 700, or 1000 batches produced at the first concrete production facility and/or not more than 2, 3, 4, 5, 6, 7, 8, 9, 10, 12, 15, 20, 30, 40, 50, 70, 100, 200, 500, 700, 1000, or 2000 batches produced at the first concrete production facility. If more than one concrete production facility is included in the method, the method may further include adding exogenous carbon dioxide to any number of additional batches of concrete at each of the additional facilities, determining information regarding carbon dioxide flow or quantity, transmitting information to the first processor, and processing the information, as described; for example, at least 1, 2, 3, 4, 5, 6, 7, 8, 9, 10, 12, 15, 20, 30, 40, 50, 70, 100, 200, 500, 700, or 1000 batches produced at any of the additional concrete production facilities and/or not more than 2, 3, 4, 5, 6, 7, 8, 9, 10, 12, 15, 20, 30, 40, 50, 70, 100, 200, 500, 700, 1000, or 2000 batches produced at any of the additional concrete production facilities. Each of the additional concrete production facilities may send information from one or more sensors, one or more HMIs, or a combination thereof, to the processor, to be processed as described. Information for batches, from one or a plurality of facilities, can, in some cases, be aggregated, in any suitable manner, e.g., information for batches with the same mix design, information for batches used in the same project, and the like.

In certain embodiments provided is a method comprising (i) contacting carbon dioxide with a concrete mix, a component of a concrete mix, or both, to carbonate the concrete mix, the component of the concrete mix, or both; (ii) determining an amount of carbon dioxide sequestered in the concrete mix or component of the mix, or both; and/or determining an amount of carbon dioxide avoided in the concrete mix or component of the mix, or both; and (iii) generating a greenhouse gas token to represent the amount of carbon dioxide sequestered and/or avoided in the concrete mix. In certain embodiments the method further comprises using the concrete mix as a construction material. In certain embodiments the token comprises a CO₂ emission certificate, a CO₂ emission permit, a CO₂ emission credit, a carbon offset, carbon allowance, a criteria pollutant allowance, a Verified Emissions Reductions unit (VER), a Carbon Financial Instrument (CFI), a European Union Allowance (EUA), a Certified Emission Reduction unit (CER), an Emission Reduction Unit (ERU), a Voluntary Carbon Unit, or a tipping fee. In certain embodiments the method further comprises exchanging the certificate for a greenhouse gas emission credit or a carbon offset. In certain embodiments determining the amount of carbon dioxide sequestered and/or avoided comprises determining one or more of an amount of carbon dioxide added to the concrete mix or component of the concrete mix or both, a weight of cement used in the concrete mix, a weight of aggregates used in the concrete mix, a temperature for a part of a system for delivering carbon dioxide to the concrete mix or component of the concrete mix, a pressure for a part of a system for delivering carbon dioxide to the concrete mix or component of the concrete mix, or a combination thereof. In certain embodiments the method further comprises representing the amount of carbon dioxide sequestered and/or avoided, and/or a carbon credit based on the amount, in visual form.

In certain embodiments provided is a greenhouse gas sequestration and avoidance system comprising (i) a concrete batching facility configured to produce batches of concrete; (ii) one or more apparatuses for adding carbon dioxide to one or more of the batches of concrete and/or to one or more components of the one or more batches of concrete; (iii) a system to monitor the amount of carbon dioxide added to the one or more batches or the one or more components and to transmit information about the amount to a processor; (iv) optionally, a system to monitor one or more characteristics of the concrete batch, comprising weight of cement added, weight of aggregates added, a temperature for a part of a system for delivering carbon dioxide to the concrete mix or component of the concrete mix, a pressure for a part of a system for delivering carbon dioxide to the concrete mix or component of the concrete mix, or a combination thereof, and to transmit information about the weight, temperature, and/or pressure to the processor; and (v) the processor, wherein the processor is configured to receive the information about the amount, weight, temperature, and/or pressure and to process the information and determine an amount of carbon dioxide sequestered, an amount of carbon dioxide avoided, or a combination thereof, for one or more batches of concrete produced at the facility. Monitoring systems, processors, and the like can be any suitable component, such as those described elsewhere herein.

In certain embodiments provided is a method for constructing a carbon-reduced structure, comprising: (i) creating a construction structure comprising a carbon-reduced concrete according to a construction plan, wherein the carbon-reduced concrete is produced at a facility that carbonates the concrete and/or one or more components of the concrete; (ii) calculating an amount of carbon dioxide sequestered and/or avoided by the carbon-reduced concrete compared to the same concrete without carbonation; and (iii) calculating a total amount of carbon dioxide sequestered and/or avoided for the carbon-reduced structure based, at least in part, on the amount of carbon dioxide sequestered and/or avoided by the reduced-carbon concrete used in the structure. In certain embodiments the method further comprises: calculating a carbon credit payback of the reduced-carbon construction based on the carbon reduction. In certain embodiments provided is a structure constructed according to the method described previously in this paragraph.

In certain embodiments provided is an apparatus for constructing a carbon-reduced structure, comprising: a hardware processor; and a memory storing instructions that, when executed by the hardware processor, cause the hardware processor to provide information for: creating a reduced-carbon structure according to a construction plan utilizing reduced-carbon concrete; calculating an amount of carbon sequestered and/or avoided in the reduced-carbon structure due to the use of the reduced-carbon concrete; performing the construction of the reduced-carbon structure; and obtaining a carbon reduction based on the calculation. In certain embodiments the hardware processor is further configured for: calculating a carbon credit payback of the reduced carbon structure based on the carbon reduction.

In certain embodiments provided is a method comprising the steps of: (i) forming a first cementitious mixture comprising a first cement weight of ordinary Portland cement; a first weight of water; and, optionally a first weight of aggregate; (ii) determining a first compressive strength of the first cementitious mixture; (iii) forming a second cementitious mixture comprising a second cement weight of the ordinary Portland cement; a second weight of water and, optionally, a second weight of aggregate, and carbon dioxide; (iv) determining a second compressive strength of the second cementitious mixture; and (v) if the second compressive strength is greater than ninety percent of the first compressive strength, creating a carbon impact number for the second cementitious mix. In certain embodiments the method further includes dividing the first cement weight by the second cement weight to calculate a carbon impact number. In certain embodiments the method further comprises the step of multiplying the carbon impact number by a number of tons of the cementitious mixture to calculate a carbon credit value.

In certain embodiments provided is system for determining carbon dioxide avoidance at a concrete production facility, comprising (i) at least one sensor to measure weight of cement added to one or more carbonated concrete batches at the facility; (ii) at least one apparatus to add carbon dioxide to a component of the carbonated concrete batches, or to add carbon dioxide to a mixing concrete batch, or both; (iii) at least one processor to accept the sensor outputs and process the sensor outputs, to determine an amount of carbon dioxide avoided in the one or more carbonated concrete batches; and (iv) at least one display module to display at least one of the processed sensor outputs.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A. A system comprising
(i) a first concrete production facility, wherein
(a) the first concrete production facility comprises a first apparatus to add exogenous carbon dioxide to a component of a first batch of concrete, the first batch of concrete, or both, produced at the facility,
(b) a first system to determine information regarding carbon dioxide flow and/or quantity added to the component of the first batch of concrete, carbon dioxide flow and/or quantity added to the first batch of concrete in the first apparatus, a mix design for the first batch of concrete, or a weight of cement used in the first batch of concrete, or a combination thereof, and
(c) a first transmitter to transmit the information to a first processor; and
(ii) the first processor, wherein the first processor
(a) receives inputs from the system for determining information in the first concrete production facility; and
(b) processes the inputs to determine an amount of carbon dioxide sequestered and/or offset for the batch of concrete.

2. The system of claim 1 wherein the component of the first batch of concrete comprises mix water, aggregates, supplementary cementitious material, cement prior to addition to the mix, or a combination thereof.

3. The system of claim 2 wherein the component comprises concrete mix water comprising carbonated wash water from the concrete production facility.

4. The system of claim 1 wherein the first processor (iii) sends the output of step (ii)(c) to a first system to provide a representation of the carbon dioxide sequestered and/or offset to a user.

5. The system of claim 1 wherein the processor further determines a carbon credit or partial credit based, at least in part, on the information from A(ii)(b).

6. The system of claim 1 wherein the first system for determining information comprises at least one sensor for sensing information regarding carbon dioxide flow and/or quantity added to the component of the first batch of concrete, carbon dioxide flow and/or quantity added to the first batch of concrete in the first apparatus, a mix design for the first batch of concrete, or a weight of cement used in the first batch of concrete.

7. The system of claim 6 wherein the first system for determining information comprises a weight sensor for sensing the weight of cement added to the first batch of concrete.

8. The system of claim 1 wherein the first system for determining information comprises a human machine interface (HMI) for entering one or more of carbon dioxide flow and/or quantity added to the component of the first batch of concrete, carbon dioxide flow and/or quantity added to the first batch of concrete in the first apparatus, a mix design for the first batch of concrete, or a weight of cement used in the first batch of concrete.

9. The system of claim 1 further comprising a second concrete production facility, different from the first concrete production facility, wherein the second concrete production facility comprises
(a) a second apparatus to add exogenous carbon dioxide to a component of a first batch of concrete, the first batch of concrete, or both, produced at the second facility,
(b) a second system to determine information regarding carbon dioxide flow and/or quantity added to the component of the first batch of concrete, carbon dioxide flow and/or quantity added to the first batch of concrete in the first apparatus, a mix design for the first batch of concrete, or a weight of cement used in the first batch of concrete, and
(c) a first transmitter to transmit the information from the second facility to a second processor.

10. The system of claim 9 wherein the first and second processors are the same.

11. The system of claim 1 wherein the first processor further receives inputs regarding market conditions for carbon credits, regulatory information, or a combination thereof.

12. The system of claim 1 wherein the first processor further receives inputs regarding the use of the first batch of concrete.

13. The system of claim 1 wherein the first processor receives inputs regarding transportation of one or more components of the first batch of concrete, transportation of the first batch of concrete to its site of use, or both.

14. A method comprising
(i) adding exogenous carbon dioxide to a component of a first batch of concrete, the first batch of concrete, or both, produced at a first concrete production facility;
(ii) determining information regarding carbon dioxide flow and/or quantity added to the component of the first batch of concrete, carbon dioxide flow and/or quantity added to the first batch of concrete, a mix design for the first batch of concrete, or a weight of cement used in the first batch of concrete, and
(iii) transmitting the information to a first processor; and
(iv) processing the information at the first processor to determine an amount of carbon dioxide sequestered and/or offset for the first batch of concrete.

15. The method of claim 14 the component of the first batch of concrete comprises mix water, aggregates, supplementary cementitious material, cement prior to addition to the mix, or a combination thereof.

16. The method of claim 15 wherein the component of the first batch of concrete comprises mix water comprising carbonated wash water from the concrete production facility.

17. The method of claim 14 wherein the first processor (iii) sends the output of step (iv) to a first system to provide a representation of the carbon dioxide sequestered and/or offset to a user.

18. The method of claim 14 wherein the processor further determines a carbon credit or partial credit based, at least in part, on the information from step (iv).

19. The method of claim 14 wherein the first system for determining information receives information from at least one sensor for sensing information regarding carbon dioxide flow and/or quantity added to the component of the first batch of concrete, carbon dioxide flow and/or quantity added to the first batch of concrete in the first apparatus, a mix design for the first batch of concrete, or a weight of cement used in the first batch of concrete.

20. The method of claim 19 wherein the first system for determining information comprises a weight sensor for sensing the weight of cement added to the first batch of concrete.

21. The method of claim 14 wherein the first system for determining information comprises a human machine interface (HMI) for entering one or more of carbon dioxide flow and/or quantity added to the component of the first batch of concrete, carbon dioxide flow and/or quantity added to the first batch of concrete in the first apparatus, a mix design for the first batch of concrete, or a weight of cement used in the first batch of concrete.

22. The method of claim 14 further comprising
(i) adding exogenous carbon dioxide to a component of a first batch of concrete, the first batch of concrete, or both, produced at a second concrete facility, different from the first concrete production facility,
(ii) determining information regarding carbon dioxide flow and/or quantity added to the component of the first batch of concrete at the second concrete production facility, carbon dioxide flow and/or quantity added to the first batch of concrete at the second concrete production facility, a mix design for the first batch of concrete at the second concrete production facility, or a weight of cement used in the first batch of concrete at the second concrete production facility, and
(c) transmitting the information from the second concrete production facility to a second processor.

23. The method of claim 22 wherein the first and second processors are the same.

24. The method of claim 22 wherein the first and second concrete production facilities are owned, operated, and/or controlled by the same entity.

25. The method of claim 14 wherein the first processor further receives inputs regarding market conditions for carbon credits, regulatory information, or a combination thereof.

26. The method of claim 14 wherein the first processor further receives inputs regarding the use of the first batch of concrete.

27. The method of claim 14 wherein the first processor receives inputs regarding transportation of one or more components of the first batch of concrete, transportation of the first batch of concrete to its site of use, energy use and/or carbon dioxide production at the first concrete production facility during production of the first concrete batch, or a combination thereof.

28. The method of claim 14 further comprising
(i) adding exogenous carbon dioxide to a component of a second batch of concrete, different from the first batch of concrete, the second batch of concrete, or both, produced at a first concrete production facility;
(ii) determining information regarding carbon dioxide flow and/or quantity added to the component of the second batch of concrete, carbon dioxide flow and/or quantity added to the second batch of concrete, a mix design for the second batch of concrete, or a weight of cement used in the second batch of concrete, and
(iii) transmitting the information to the first processor; and
(iv) processing the information at the first processor to determine an amount of carbon dioxide sequestered and/or offset for the second batch of concrete.

29. A network comprising
(i) a plurality of concrete production facilities, wherein each facility comprises
(a) an apparatus to add exogenous carbon dioxide to a component of a first batch of concrete, the first batch of concrete, or both, produced at the facility,
(b) a system to determine information regarding carbon dioxide flow and/or quantity added to the component of the first batch of concrete, carbon dioxide flow and/or quantity added to the first batch of concrete in the apparatus, a mix design for the first batch of concrete, or a weight of cement used in the first batch of concrete, and
(c) a transmitter to transmit the information to a processor;
(ii) the processor, which is configured to
(a) receive the information from each of the plurality of concrete production facilities,
(b) process the information for each facility to determine an amount of carbon dioxide sequestered and/or avoided for the first batch of concrete produced at each facility.
